(19) United States
(12) Patent Application Publication
(10) Pub. No.: US 2006/0140865 A1
(43) Pub. Date: Jun. 29, 2006

(54) Nano-micelle carrier used in near infrared image detection and detection method thereof

(75) Inventors: Wen Hsiang Chang, Taipei City (TW); Chao-Hung Kao, Taipei (TW); Chi-Min Chau, Taichung County (TW); Chin-I Lin, Tainan County (TW); Shian-Jy Jass Wang, Hsinchu County (TW)

Correspondence Address: BIRCH STEWART KOLASCH & BIRCH, PO BOX 747, FALLS CHURCH, VA, 22040-0747, US

(73) Assignee: Industrial Technology Research Institute

(21) Appl. No.: 11/317,077

(22) Filed: Dec. 27, 2005

(30) Foreign Application Priority Data
Dec. 29, 2004 (TW) 93141163

(51) Int. Cl. A61K 49/00

(52) U.S. Cl. 424/9.6; 977/927

(57) ABSTRACT

A nano-micelle carrier used in near infrared image detection. The carrier includes a nano-micelle comprising a plurality of block copolymers having critical micelle concentration (CMC) and a near infrared dye grafted onto the micelle surface, wherein the carrier has a hydrophobic interior and a hydrophilic surface. The invention also provides a method of detecting a near infrared image.

### BACKGROUND

The invention relates to a nano-micelle carrier, and more specifically to a nano-micelle carrier used in near infrared image detection and a detection method thereof.

When an animal tissue image is analyzed, absorption wavelengths of other materials within the sample, such as hemoglobin, water, or phospholipid, must be considered simultaneously, because emission light of a target tissue may be absorbed thereby, without obtaining an exact and clear detection image, for example, hemoglobin absorbs visible light and water and lipids may absorb infrared light. Thus, if a target cell emits visible light or infrared light, it cannot pass through blood or tissues, resulting in a deteriorated detection image. Near infrared (NIR) light, however, is seldom absorbed by these materials. Therefore, quality of detection image can be greatly improved by controlling wavelength of emission light of target cell in NIR range.

Harvard Medicine Center provides a method of detecting a near infrared image. First, a NIR dye transporting carrier is prepared, for example, by grafting onto a nanoparticle of iron oxide conjugate with dextrane by a peptide. An NIR image of a target cell is then detected. When the NIR dye bonds to a nanoparticle of iron oxide conjugate with dextrane, it is quenched so that the carrier does not emit light until entering the target cell. After the nanoparticle carrier enters the target cell, the peptide inserted between the NIR dye and the nanoparticle is cut by enzyme. The NIR dye is then released and dequenched to emit NIR light. The location of the target cell within an animal can be obtained by detecting the NIR light.

### SUMMARY

The invention provides a nano-micelle carrier used in near infrared image detection, comprising a nano-micelle comprising a plurality of copolymers having critical micelle concentration (CMC) and a near infrared dye grafted onto the nano-micelle surface or interior, wherein the nano-micelle has a hydrophobic interior and a hydrophilic surface.

The invention also provides a method of detecting a near infrared image, comprising dosing a subject with a nano-micelle carrier comprising a plurality of copolymers having critical micelle concentration (CMC) and a near infrared dye grafted onto the nano-micelle surface or interior. After the nano-micelle carrier is devoured by a target cell, the near infrared dye is dequenched due to disintegration of the nano-micelle carrier. Next, the subject is irradiated with an excitation light to excite the near infrared dye, emitting near infrared light. Finally, a near infrared image of the subject is obtained by analyzing the near infrared light to acquire a location signal of the target cell inside the subject.

A detailed description is given in the following embodiments with reference to the accompanying drawings.

### BRIEF DESCRIPTION OF THE DRAWINGS

Embodiments of the invention can be more fully understood by reading the subsequent detailed description and examples with references made to the accompanying drawings, wherein:

FIG. 1 shows a nono-micelle carrier structure of the invention.

### DETAILED DESCRIPTION

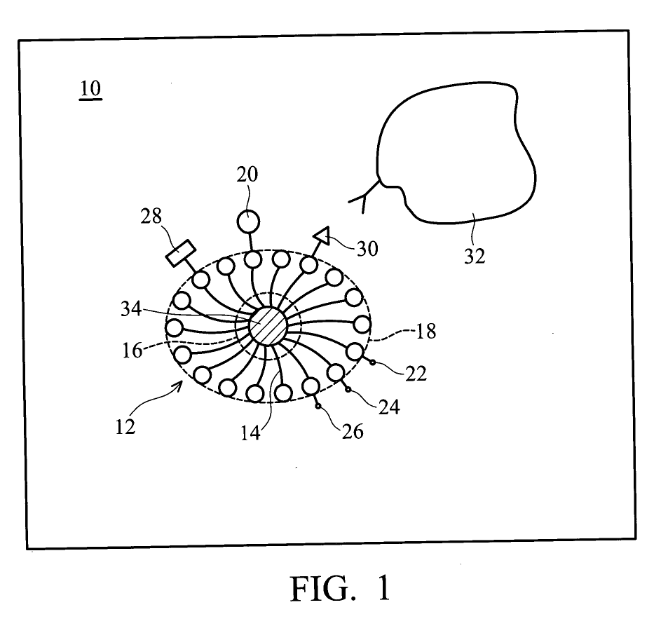

The nano-micelle carrier structure provided by the invention is disclosed in FIG. 1. The carrier 10 comprises a nano-micelle 12 comprising a plurality of copolymers 14 having critical micelle concentration (CMC), and a near infrared dye 20 grafted onto the nano-micelle surface, wherein the nano-micelle 12 has a hydrophobic interior 16 and a hydrophilic surface 18. The surface 18 of the nano-micelle has hydroxyl groups 22, amino groups 24, or carboxyl groups 26, a near infrared quencher 28 used to increase the quench effect, or a ligand 30 grafted thereon, wherein the ligand 30, such as folic acid, recognizes a target cell 32, such as a tumor cell in the subject. Additionally, the nano-micelle carrier 10 can be a drug delivery carrier when a lipophilic drug 34 is packaged inside the micelle 12.

The copolymers comprise diblock or tribolck copolymers. The near infrared dye comprises a fluorescent or phosphorescent dye. The nano-micelle has a diameter of about 10˜300 nm.

The preparation of the nano-micelle carrier is described as follows. First, a block copolymer, such as PEG-PCL, is prepared. Next, the block copolymer is dissolved in a solvent, such as THF. The resulting solution is then injected into deionized water with ultrasonic agitation to form a nano-micelle by assembling the block copolymers. After the solvent is removed, a near infrared dye is added to graft to the nano-micelle. Finally, near infrared dye without grafting is removed to form a nano-micelle carrier.

Block copolymers provide critical micelle concentration (CMC), that is, when the block copolymer concentration exceeds CMC, the block copolymers may be assembled to form a micelle. When the block copolymer concentration is lower than CMC, such as entering a cell, the block copolymer may be disintegrated, returning to the original dispersed form. The invention provides a micelle capable of structural alteration in various environments to control emission mechanism of near infrared dye.

The invention provides a method of detecting near infrared image. First, an subject is dosed with a nano-micelle carrier comprising a plurality of copolymers having critical micelle concentration (CMC) and a near infrared dye grafted onto the nano-micelle surface. After the nano-micelle carrier is devoured by a target cell, the near infrared dye is dequenched due to disintegration of the nano-micelle carrier. Next, the subject is irradiated with an excitation light to excite the near infrared dye, emitting near infrared light. Finally, a near infrared image of the subject is analyzed to obtain a location of the target cell in the subject.

When NIR dye is grafted onto a micelle, quenching may occur. To increase the quench effect, the invention further provides a quencher grafted thereon. After the micelle is devoured by a target cell, the micelle disintegrates and is dequenched due by polymer concentration within the cell lower than CMC. At this time, the target cell is irradiated with an excitation light. The excited NIR dye may then emit a NIR light detectable by an external detector.

### EXAMPLE

Preparation of nano-micelle carrier

First, a PEG-PCL diblock copolymer was prepared with PEG at a molecular weight of about 2000, and PCL about 2300. The diblock copolymer had a CMC of about 0.25 mg/ml.

Next, transformation of terminal functional groups of the PEG-PCL copolymer was performed, that is, transforming hydroxyl groups thereof into amino groups to improve bonding efficiency between a subsequently added NIR dye and a subsequently formed micelle. First, 2 g PEG-PCL was dissolved in 2 ml dichloromethane. 0.2 ml TMSI was then added to react. After dichloromethane was removed by rotary evaporation, the PEG-PCL copolymer was dissolved in THF. Next, Na₂S₂O₅ was added to stop the reaction. Na₂S₂O₅ was prepared by dissolving 10% Na₂S₂O₅ in 0.1 N HCl. Next, hexane was added to recrystallize the PEG-PCL copolymer. The crystalline solid was then dialyzed, purified, and freeze-dried. Next, the PEG-PCL copolymer was dissolved in DMF. Next, triethylamine was added to the DMF solution, wherein the molar ratio of triethylamine and the PEG-PCL copolymer was 3:1. Next, 3-bromopropylamine hydrobromide was added, wherein molar ratio of 3-bromopropylamine hydrobromide and the PEG-PCL copolymer was 3:1, at reaction temperature of 50° C. After dialyzing, the PEG-PCL copolymer was collected by freeze-drying, and the transformation of a hydroxyl group on the PEG terminal to a amino group was completed.

Next, 20, 40, and 60 mg/ml PEG-PCL copolymers were dissolved in THF, respectively. The forgoing solutions were then respectively added deionized water with ultrasonic agitation for 2 min to form a nano-micelle by assembling the block copolymers, wherein the volume ratios of THF and deionized water were 1:1, 1:2, and 1:3, respectively. After THF was removed by dialyzing, NIR dye was added to graft with the nano-micelle. Finally, NIR dye without grafting was removed by dialyzing to form a nano-micelle carrier. The nano-micelle has a diameter of about 35.5˜38.5 nm.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A nano-micelle carrier used in near infrared image detection, comprising:
a nano-micelle comprising a plurality of block copolymers having critical micelle concentration (CMC), the nano-micelle comprising a hydrophobic interior and hydrophilic surface; and
a near infrared dye grafted onto the nano-micelle surface.

2. The nano-micelle carrier as claimed in claim 1, wherein the block copolymers comprise diblock or triblock copolymers.

3. The nano-micelle carrier as claimed in claim 1, wherein the nano-micelle surface has hydroxyl, amino, or carboxyl groups thereon.

4. The nano-micelle carrier as claimed in claim 1, wherein the near infrared dye comprises a fluorescent or phosphorescent dye.

5. The nano-micelle carrier as claimed in claim 1, further comprising a near infrared quencher grafted onto the nano-micelle surface.

6. The nano-micelle carrier as claimed in claim 1, further comprising a ligand grafted onto the nano-micelle surface.

7. The nano-micelle carrier as claimed in claim 6, wherein the ligand recognizes a target cell of a subject.

8. The nano-micelle carrier as claimed in claim 6, wherein the ligand comprises folic acid.

9. The nano-micelle carrier as claimed in claim 7, wherein the target cell comprises a tumor cell.

10. The nano-micelle carrier as claimed in claim 1, further comprising a lipophilic drug packaged inside the micelle.

11. The nano-micelle carrier as claimed in claim 10, wherein the nano-micelle carrier is a drug delivery carrier.

12. The nano-micelle carrier as claimed in claim 1, wherein the nano-micelle has a diameter of about 10˜300 nm.

13. A method of detecting a near infrared image, comprising:
dosing a subject with a nano-micelle carrier as claimed in claim 6, resulting in dequenching of the near infrared dye due to disintegration of the nano-micelle carrier when devoured by a target cell;
irradiating the subject with an excitation light to excite the near infrared dye to emit near infrared light; and
detecting a near infrared image of the animal to obtain a location of the target cell within the subject.
